(19) United States
(12) Patent Application Publication
(10) Pub. No.: US 2021/0251547 A1
(43) Pub. Date: Aug. 19, 2021

(54) MEASUREMENT DEVICE AND MEASUREMENT SYSTEM

(71) Applicant: Murata Manufacturing Co., Ltd., Nagaokakyo-shi (JP)

(72) Inventors: Daiki Ogata, Nagaokakyo-shi (JP); Yoshie Kuramochi, Nagaokakyo-shi (JP)

(21) Appl. No.: 17/177,473

(22) Filed: Feb. 17, 2021

(30) Foreign Application Priority Data
Feb. 17, 2020 (JP) 2020-024544
Aug. 6, 2020 (JP) 2020-133957

(51) Int. Cl. A61B 5/277; A61B 5/251; G01N 27/22

(52) U.S. Cl. CPC A61B 5/277; G01N 27/223; A61B 5/251

(57) ABSTRACT

A measurement device includes a biosensor that acquires biological information, a pressing force detection component that detects a pressing force produced when the biosensor makes contact with a part of a living body to be measured, and a processor that converts the biological information to a first measurement value, calculates a second measurement value by correcting the first measurement value based on the pressing force, and outputs the second measurement value.

### CROSS-REFERENCE TO RELATED APPLICATIONS

The present application claims priority to Japanese Application No. 2020-024544, filed on Feb. 17, 2020, and to Japanese Application No. 2020-133957, filed Aug. 6, 2020, the entire contents of each of which are incorporated herein by reference.

### TECHNICAL FIELD

The present disclosure relates to measurement devices and measurement systems that collect information via a sensor that is comes in physical contact with a target.

### BACKGROUND

Measurement systems that collect biological information come in many forms. For example, a measurement system may be an instrument for measuring water content in an oral cavity. Typically, such an instrument is made up of a sensor and a measurement component including the sensor. The sensor detects the water content of a target to be measured by bringing the sensor directly into contact with the target or by bringing the sensor into contact with the target with a plastic film or the like interposed in between. The measurement component then generates a measurement value (e.g., an amount of water content).

In such systems, it is difficult to obtain an accurate measurement value when the contact between the sensor and a target is inadequate. Therefore, when a measurement is performed, in order to ensure the contact between a target and the sensor, the sensor is firmly pressed against the target to be measured. In some cases, a measurement is initiated only when the pressing force of the sensor against the target exceeds a predetermined threshold value. This predetermined threshold value is the value of the pressing force, a level of which ensures the contact between the target and the sensor.

However, even in the case where the pressing force of the sensor against a target exceeds the predetermined threshold value, there is an issue of variations in measurement value depending on the size of the pressing force. For example, a firm press and a light press, even in the same contact location of the target, will cause conventional systems to output different measurement values. Thus, there is a need for consistency in measurements.

### SUMMARY

To address the shortcomings of conventional measurement systems, the present disclosure describes a measurement system that corrects a measurement value according to the size of a pressing force produced when a biosensor comes in contact with a part of a living body to be measured.

A measurement device according to one aspect of the present disclosure includes a biosensor that acquires biological information, a pressing force detection component that detects a pressing force produced when the biosensor makes contact with a part of a living body to be measured, and a processor that converts the biological information to a first measurement value, calculates a second measurement value by correcting the first measurement value based on the pressing force, and outputs the second measurement value.

A measurement system according to one aspect of the present disclosure includes a measurement device, and a processing device that communicates with the measurement device, wherein the measurement device includes a biosensor that acquires biological information, a pressing force detection component that detects a pressing force produced when the biosensor makes contact with a part of a living body to be measured, a processor that converts the biological information to a first measurement value, calculates a second measurement value by correcting the first measurement value based on the pressing force, and outputs the second measurement value, and a first communication component that transmits the information on the second measurement value to the processing device, and the processing device includes a second communication component that receives the information on the second measurement value from the first communication component of the measurement device, and a calculation component that calculates an amount of a measuring target based on the information on the second measurement value.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and form a part of this specification, illustrate one or more example aspects of the present disclosure and, together with the detailed description, serve to explain their principles and implementations.

FIG. 1 is a schematic perspective view of an exemplary measurement device according to a first embodiment of the present disclosure;

FIG. 2 is a schematic view illustrating an internal configuration of an exemplary measurement device according to the first embodiment of the present disclosure;

FIG. 3 is a block diagram illustrating a schematic configuration of an exemplary measurement device according to the first embodiment of the present disclosure;

FIG. 4 is a schematic enlarged bottom view of a sensor in an exemplary measurement device according to the first embodiment of the present disclosure;

FIG. 5 is a table illustrating examples of pressing force, first measurement value, correction factor, and second measurement value;

FIG. 6 is a diagram illustrating a method of calculating a correction factor;

FIG. 7A is a diagram illustrating a method of calculating an average value of pressing forces;

FIG. 7B is a diagram illustrating a method of calculating an average value of pressing forces;

FIG. 8 is a flowchart illustrating an example of operation of a measurement device according to the first embodiment of the present disclosure;

FIG. 9 is a schematic view illustrating an example of a situation where a measurement device is being used according to the first embodiment of the present disclosure;

FIG. 10 is a view illustrating an internal configuration of a measurement device in a modified example according to the first embodiment of the present disclosure;

FIG. 11 is a block diagram illustrating a schematic configuration of a measurement device in a modified example according to the first embodiment of the present disclosure;

FIG. 12 is a block diagram illustrating a schematic configuration of another exemplary measurement device according to a second embodiment of the present disclosure;

FIG. 13 is a flowchart illustrating an example of operation of the another exemplary measurement device according to the second embodiment of the present disclosure;

FIG. 14 is a block diagram illustrating a schematic configuration of an exemplary measurement system according to a third embodiment of the present disclosure; and

FIG. 15 is a flowchart illustrating an example of operation of an exemplary measurement system according to the third embodiment of the present disclosure.

### DETAILED DESCRIPTION

A measurement device according to one aspect of the present disclosure includes a biosensor that acquires biological information, a pressing force detection component that detects a pressing force produced when the biosensor makes contact with a part of a living body to be measured, and a processor that converts the biological information to a first measurement value, calculates a second measurement value by correcting the first measurement value based on the pressing force, and outputs the second measurement value. Such configuration improves measurement accuracy.

In some aspects, the processor may increase a correction amount of the first measurement value as the pressing force increases. Such configuration further improves the measurement accuracy.

In some aspects, the processor may start a measurement process when the pressing force is equal to or greater than a first threshold value. Such configuration enables to start the measurement process after ensuring the contact between the biosensor and the part of a living body to be measured. This further improves the measurement accuracy.

In some aspects, the processor may correct the first measurement value based on an average value of the pressing forces detected during a predetermined time period after the start of the measurement process. Such configuration further improves the measurement accuracy.

In some aspects, the processor may correct the first measurement value based on the average value when the average value is equal to or greater than a second threshold value and equal to or less than a third threshold value. Such configuration further improves the measurement accuracy.

In some aspects, the measurement device may further include a housing having a lengthwise direction, the housing storing therein the biosensor, the pressing force detection component, and the processor, wherein the housing has a sensor provided on one end along the lengthwise direction of the housing, a grip provided on another end along the lengthwise direction, and a probe formed in a rod-like shape, the grip connecting the sensor and the grip, the biosensor is arranged in the sensor, the pressing force detection component is arranged in the sensor or the probe, and the processor is arranged in the probe. Such configuration facilitates an accurate detection of the pressing force, which further improves the measurement accuracy.

In some aspects, the biosensor may have a detection surface that acquires the biological information, and the pressing force detection component may be arranged inside the sensor and be arranged on an inner side of an outer perimeter of the detection surface when viewed from a direction orthogonal to the detection surface. Such configuration further facilitates a more accurate detection of the pressing force, which further improves the measurement accuracy.

In some aspects, the biosensor may be an electrostatic capacitance sensor that detects electrostatic capacitance, and the processor may perform a conversion process that converts an electrostatic capacitance detected by the electrostatic capacitance sensor into a frequency. Such configuration further improves the measurement accuracy.

In some aspects, the measurement device may further include a calculation component that calculates an amount of a measuring target based on the second measurement value. Such configuration enables the calculation of the amount of a measuring target in the measurement device.

In some aspects, the amount of the measuring target may be an amount of water content. Such configuration enables measuring of the amount of water content as the amount of the measuring target.

In some aspects, the pressing force detection component may be a piezoelectric pressure sensor. Such configuration further facilitates a more accurate detection of the pressing force. This further improves the measurement accuracy.

In some aspects, the measurement device may further include a notification component that gives notice of information, wherein the processor determines whether or not the pressing force is in a range between predetermined threshold values and outputs information on a determination result to the notification component. Such configuration improves usability of the measurement device.

In some aspects, the part of a living body to be measured is a part to be measured in an oral cavity. Such configuration enables measuring a state in the oral cavity with a high degree of accuracy.

A measurement system according to one aspect of the present disclosure includes a measurement device and a processing device that communicates with the measurement device, wherein the measurement device includes a biosensor that acquires biological information, a pressing force detection component that detects a pressing force produced when the biosensor makes contact with a part of a living body to be measured, a processor that calculates a second measurement value and outputs information on the second measurement value, the second measurement value being calculated by correcting a first measurement value based on the pressing force, the first measurement value being obtained based on the biological information, and a first communication component that transmits the information on the second measurement value to the processing device, and the processing device includes a second communication component that receives the information on the second measurement value from the first communication component of the measurement device, and a calculation component that calculates an amount of a measuring target based on the information on the second measurement value. Such configuration further improves measurement accuracy.

Next, an embodiment of the present disclosure is described with reference to the accompanying drawings. Note that the following descriptions are merely examples in essence and are not intended to limit the present disclosure, its application, or its usage. Furthermore, the drawings are schematic and not necessarily matched with actual ones in the ratio among dimensions.

### Embodiment 1

### Overall Configuration

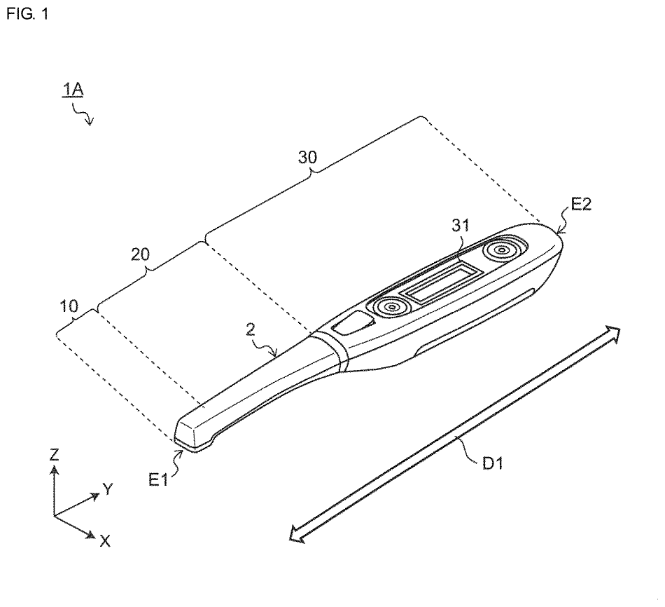

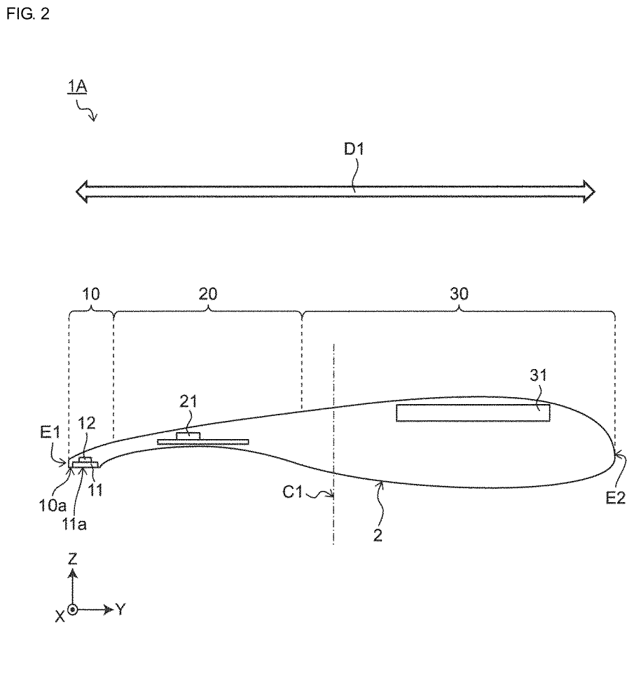

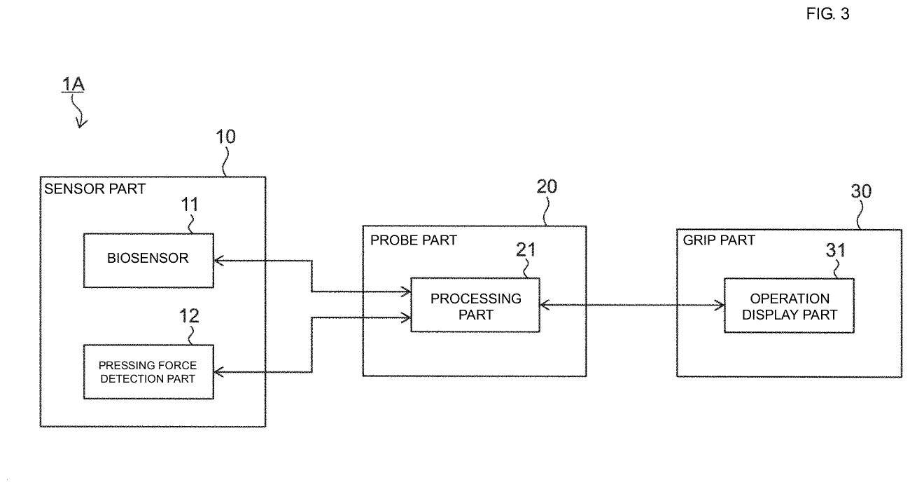

FIG. 1 is a schematic perspective view of an example of a measurement device 1A of the first embodiment according to the present disclosure. FIG. 2 is a schematic view illustrating an internal configuration of the example of the measurement device 1A of the first embodiment according to the present disclosure. FIG. 3 is a block diagram illustrating a schematic configuration of the example of the measurement device 1A of the first embodiment according to the present disclosure. The X, Y, and Z direction in the drawings represent the width direction, the length direction, and the height direction of the measurement device 1A, respectively.

In embodiment 1, an example is described in which the measurement device 1A is a device for measuring an inside of the oral cavity. Furthermore, in embodiment 1, a measuring target of the measurement device 1A is water content, and an example is described in which the amount of water content in the oral cavity is measured using the measurement device 1A.

### Exterior Shape

An exterior shape of the measurement device 1A is now described. As illustrated in FIG. 1 and FIG. 2, the measurement device 1A includes a housing 2. The housing 2 has a substantially rod-like shape with a lengthwise direction D1. Specifically, the housing 2 has a sensor 10, a probe 20, and a grip 30.

The sensor 10 is a component that makes contact with a part of a living body to be measured. The part of a living body to be measured is a part to be measured in the oral cavity. The part to be measured in the oral cavity is, for example, a tongue. The sensor 10 is provided at one end E1 of the measurement device 1A in the lengthwise direction D1. External dimensions of the sensor 10 are designed to be smaller than those of the probe 20 and the grip 30. For example, the dimensions of the sensor 10 in the X direction and the Y direction are designed to be smaller than those of the probe 20 and the grip 30.

The sensor 10 has a contact surface 10a that makes contact with a part of a living body to be measured. The contact surface 10a is provided on the one end E1 side of the housing 2 in the lengthwise direction D1 and is provided in such a way that the contact surface 10a spreads out in the directions (X and Y directions) that cross an end face on the one end E1 side.

The probe 20 connects the sensor 10 and the grip 30. The probe 20 is formed in a substantially rod-like shape. The dimensions of the probe 20 in the X direction and the Z direction decrease from the grip 30 to the sensor 10. That is to say, the probe 20 has a shape that tapers from the grip 30 to the sensor 10.

The grip 30 is a component that a user grasps. The grip 30 is provided on the other end E2 side of the measurement device 1A in the lengthwise direction D1. The grip 30 is formed in a substantially rod-like shape. External dimensions of the grip 30 are designed to be larger than those of the sensor 10 and the probe 20. For example, the dimensions of the grip 30 in the X, Y, and Z directions are designed to be larger than those of the sensor 10 and the probe 20.

The housing 2 is made of, for example, a resin. A component of the housing 2 may be made of a metal. Alternatively, the whole of the housing 2 may be made of a metal.

Next, elements that make up the measurement device 1A are described. As illustrated in FIG. 1 to FIG. 3, the measurement device 1A includes a biosensor 11, a pressing force detection component 12, a processor 21, and an operation display component 31.

Note that in embodiment 1, the example is described in which the measurement device 1A includes the operation display component 31. However, the present embodiment is not limited to this example. It is noted that the operation display component 31 is not an essential element and may be included in another device different from the measurement device 1A.

### Biosensor

The biosensor 11 acquires biological information. The biological information is a variety of physiological and anatomical information that a living body produces. The biological information is, for example, information on electrostatic capacitance, resistance value, amount of water content, temperature, stiffness, heart rate, pulse, dielectric constant, cardio-electricity, myoelectricity, or the like. The biosensor 11 is brought into contact with a part to be measured in the oral cavity of a user and acquires biological information of the contacted part to be measured.

In embodiment 1, the biosensor 11 is, for example, an electrostatic capacitance sensor. The biosensor 11 is brought into contact with a part to be measured in the oral cavity and acquires information on the electrostatic capacitance. That is to say, in embodiment 1, the biological information acquired by the biosensor 11 is information on the electrostatic capacitance.

The biosensor 11 is arranged at the contact surface 10a. The biosensor 11 is arranged at the contact surface 10a on the one end E1 side of the measurement device 1A in the lengthwise direction D1. For example, the biosensor 11 is arranged in a depression component provided on the contact surface 10a side of the sensor 10 of the housing 2.

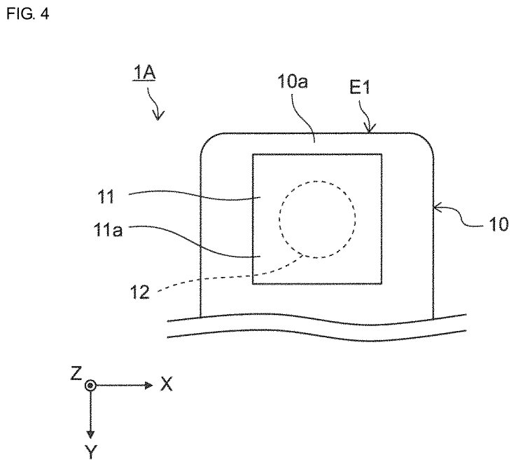

FIG. 4 is a schematic enlarged bottom view of an example of a sensor in the measurement device of an embodiment 1 according to the present disclosure. The biosensor 11 is formed in a substantially plane-like shape. Specifically, the biosensor 11 has a detection surface 11a that acquires biological information. The detection surface 11a is exposed toward the contact surface 10a side of the sensor 10. For example, the detection surface 11a is formed in a substantially rectangular shape when viewed from the height direction (Z direction) of the measurement device 1A. The detection surface 11a detects biological information by making contact with a part to be measured. That is to say, the biosensor 11 acquires biological information by bringing the detection surface 11a into contact with the part to be measured.

The biological information acquired by the biosensor 11 is transmitted to the processor 21.

### Pressing Force Detection Component

The pressing force detection component 12 detects a pressing force P produced when the biosensor 11 makes contact with a part of a living body to be measured. The pressing force P means a force that presses the biosensor 11 against a part to be measured. For example, the pressing force P means a load caused by pressing. For example, the pressing force detection component 12 is a piezoelectric pressure sensor or a strain gauge pressure sensor. The piezoelectric pressure sensor is capable of accurately measuring an extremely small force by setting the range of a charge amplifier (a processing portion for obtaining a voltage output in response to pressure). The strain gauge pressure sensor has the advantages of having no drift and a small temperature dependence.

The pressing force detection component 12 may directly detect a pressing force applied to the biosensor 11 or may indirectly detect the pressing force applied to the biosensor 11 by detecting a pressing force produced at the housing 2 when the biosensor 11 is brought into contact.

The pressing force detection component 12 is arranged in the sensor 10. The pressing force detection component 12 is arranged inside the sensor 10 and is arranged on the inner side of the outer perimeter of the detection surface 11a when viewed from the direction (Z direction) orthogonal to the detection surface 11a of the biosensor 11. Specifically, in the Z direction, the pressing force detection component 12 is arranged inside the sensor 10 on a surface of the biosensor 11 opposite the detection surface 11a.

Information on the pressing force P detected by the pressing force detection component 12 is transmitted to the processor 21.

### Processor

The processor 21 calculates a second measurement value R2 by correcting a first measurement value R1 based on the pressing force P, the first measurement value R1 being obtained based on the biological information. Furthermore, the processor 21 outputs information on the second measurement value R2.

The processor 21 acquires the first measurement value R1 based on the biological information acquired by the biosensor 11. Specifically, the processor 21 receives the biological information from the biosensor 11 and performs a conversion process that converts to information on the first measurement value R1 based on the biological information. For example, the biological information is analog information, and the information on the first measurement value R1 is digital information. In embodiment 1, the processor 21 includes a frequency conversion circuit that converts information on the electrostatic capacitance, which is the biological information acquired by the biosensor 11, into a frequency. The processor 21 receives the information on the electrostatic capacitance from the biosensor 11 and converts the electrostatic capacitance into the frequency using the frequency conversion circuit. This enables the acquisition of the frequency as the first measurement value R1.

For example, the processor 21 repeats charging and discharging of the biosensor 11 that is regarded as an electrostatic capacitance and converts into a frequency of the cycle determined by a charging-and-discharging speed.

The processor 21 receives the information on the pressing force P from the pressing force detection component 12 and calculates the second measurement value R2 by correcting the first measurement value R1 based on the pressing force P. In embodiment 1, the processor 21 includes a correction circuit that calculates the second measurement value R2 by correcting the first measurement value R1 based on the pressing force P. The processor 21 receives the information on the pressing force P from the pressing force detection component 12 and corrects the frequency based on the information on the pressing force P using the correction circuit. This enables the acquisition of the corrected frequency as the second measurement value R2.

The correction circuit increases a correction amount of the first measurement value R1 as the pressing force P increases. The correction circuit calculates the second measurement value R2 by correcting the first measurement value R1 using a correction factor Q. For example, the correction circuit calculates the second measurement value R2 by multiplying the first measurement value R1 by the correction factor Q. The correction circuit increases the correction factor Q as the pressing force P increases.

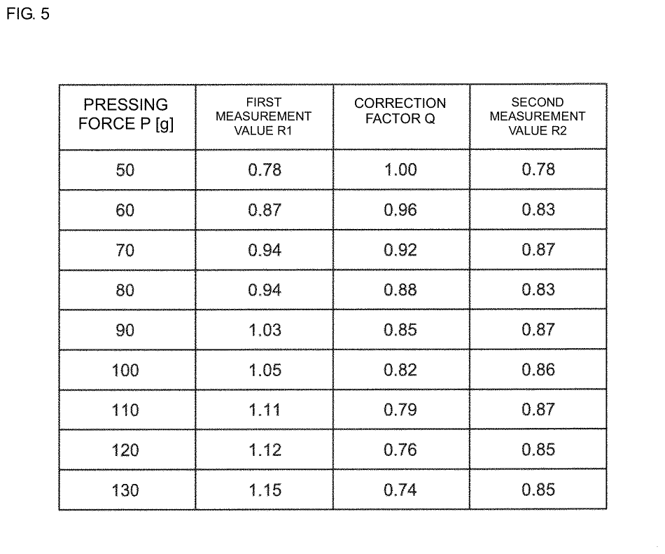

FIG. 5 is a table illustrating examples of the pressing force P, the first measurement value R1, the correction factor Q, and the second measurement value R2. Note that the examples illustrated in FIG. 5 do not include examples in which the pressing force P is smaller than 50 g because the contact between a part to be measured and the biosensor 11 cannot be ensured when the pressing force P is less than 50 g. As illustrated in FIG. 5, with an increase in the pressing force P beyond 50 g, the first measurement value R1 increases. As described above, despite of the ensured contact between a part to be measured and the biosensor 11, the first measurement value R1 varies as the pressing force P increases.

The correction factor Q is set in such a manner as to increase the correction amount as the pressing force P increases. The correction factor Q is set for each predetermined value or for each predetermined range of the pressing force P. In the example illustrated in FIG. 5, the correction factor Q is set every 10 g change in the pressing force P.

The correction circuit determines the correction factor Q based on the pressing force P and calculates the second measurement value R2 by multiplying the first measurement value R1 by the correction factor Q.

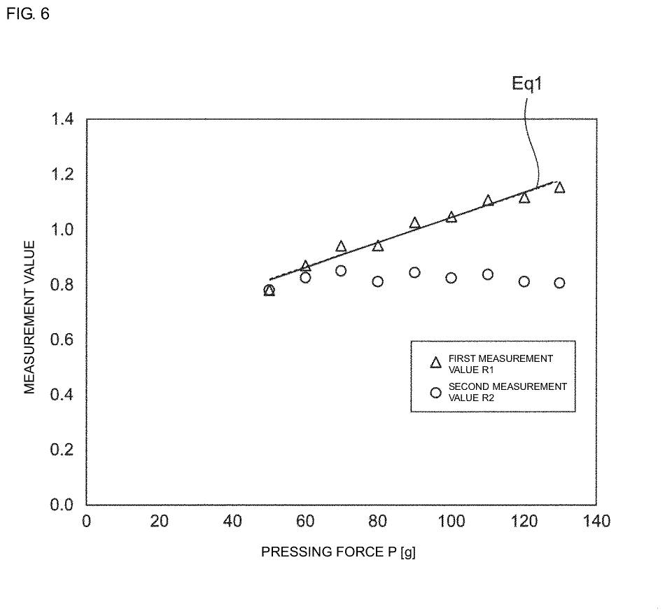

FIG. 6 is a diagram for illustrating an example of a method of calculating the correction factor Q. Note that data illustrated in FIG. 6 are the first measurement values R1 and the second measurement values R2 when only the pressing force P is varied under a predetermined condition. For example, the data illustrated in FIG. 6 may be acquired during a manufacturing step of a product.

As illustrated in FIG. 6, an approximate equation Eq1 for the pressing force P and the first measurement value R1 is calculated. For example, the approximate equation Eq1 is a linear equation. The approximate equation Eq1 can be calculated, for example, by the method of least squares. The correction factor Q is set by the ratio of each approximate value of the first measurement value R1 of the pressing force P that is not a reference value to an approximate value of the first measurement value R1 of the pressing force P that is defined as the reference value.

Note that in embodiment 1, the example is described in which the approximate equation Eq1 is a linear equation. However, the present embodiment is not limited to this example. For example, the approximate equation Eq1 may be a quadratic equation. In the case where the approximate equation Eq1 is a quadratic equation, the approximate equation Eq1 may be calculated by polynomial approximation, linear approximation, exponential approximation, repeated multiplication approximation, or logarithmic approximation.

The example illustrated in FIG. 5 and FIG. 6 illustrates the case where the first measurement value R1 at the time the pressing force P is 50 g is defined as the reference, that is, the case where the correction factor Q at the time the pressing force P is 50 g is set to “1”.

The correction circuit selects the correction factor Q that corresponds to the pressing force P and multiplies the first measurement value R1 by the correction factor Q. This enables the calculation of the second measurement value R2.

The processor 21 outputs information on the calculated second measurement value R2. For example, the processor 21 transmits the information on the calculated second measurement value R2 to a calculation component that calculates the amount of the measuring target. The calculation component may be included in the measurement device 1A or may be included in another device different from the measurement device 1A.

In embodiment 1, the processor 21 starts a measurement process when the pressing force P is equal to or greater than a first threshold value S1. The measurement process is a process for measuring the amount of the measuring target. for example, the measurement process means processes performed by the frequency conversion circuit and the correction circuit.

The processor 21 includes a determination circuit that determines whether or not the pressing force P is equal to or greater than the first threshold value S1. The processor 21 determines whether or not the pressing force P is equal to or greater than the first threshold value S1 using the determination circuit. The processor 21 starts the measurement process when the determination circuit determines that the pressing force P is equal to or greater than the first threshold value S1. The processor 21 does not start the measurement process when the determination circuit determines that the pressing force P is less than the first threshold value S1. As described above, although the processor 21 continues to receive the biological information from the biosensor 11 and the information on the pressing force P from the pressing force detection component 12, the processor does not start the measurement process unless the pressing force P becomes equal to or greater than the first threshold value S1.

For example, the first threshold value S1 is set to the value of a pressing force P, a level of which ensures the contact between a part to be measured and the biosensor 11. In the example illustrated in FIG. 5 and FIG. 6, the first threshold value S1 may be set to about 50 g.

Note that the first threshold value S1 is not limited to the above value and may be set to an arbitrary value.

In embodiment 1, the processor 21 may calculate the second measurement value R2 by correcting the first measurement value R1 based on an average value Pz of the pressing forces P detected during a predetermined time period after the start of the measurement process. The processor 21 may include a calculation circuit that calculates the average value Pz of the pressing forces P detected during a predetermined time period after the start of the measurement process. The pressing force detection component 12 detects the pressing force P during a predetermined time period after the start of the measurement process. The processor 21 calculates the average value Pz of the pressing forces P during the predetermined time period using the calculation circuit. Based on the average value Pz of the pressing forces P, the correction circuit corrects the first measurement value R1 to the second measurement value R2. For example, the correction factor Q is calculated using the average value Pz of the pressing forces P.

In embodiment 1, the processor 21 may correct the first measurement value R1 based on the average value Pz and obtains the second measurement value R2 when the average value Pz of the pressing forces P detected during a predetermined time period is equal to or greater than a second threshold value S2 and equal to or less than a third threshold value S3. For example, the second threshold value S2 is set to the value of a pressing force P, a level of which ensures the contact between a part to be measured and the biosensor 11. The second threshold value S2 may be equal to the first threshold value S1. The third threshold value S3 is set to a pressing force P, a level of which does not damage the part to be measured. For example, the second threshold value S2 may be set to about 50 g, and the third threshold value S3 may be set to about 130 g. Note that the second threshold value S2 and the third threshold value S3 are not limited the above values and may be set to arbitrary values.

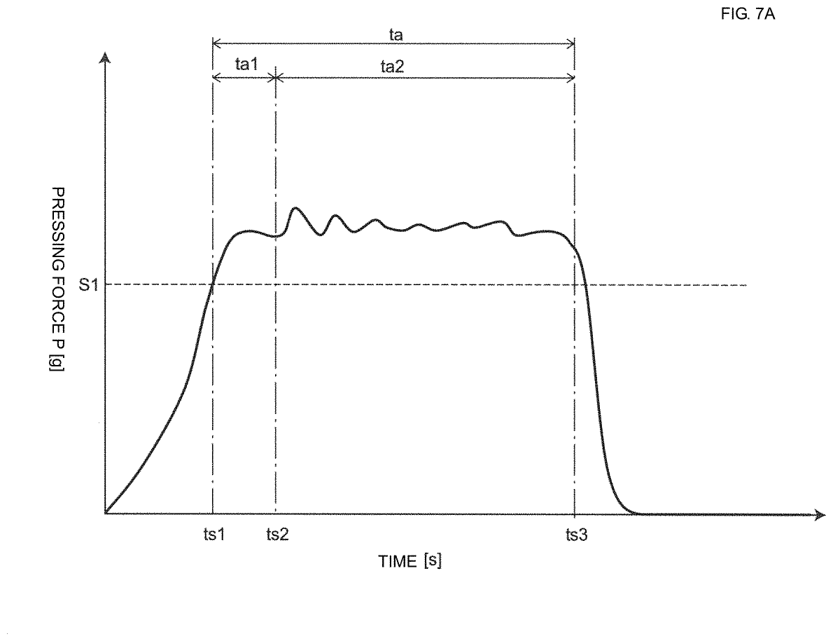

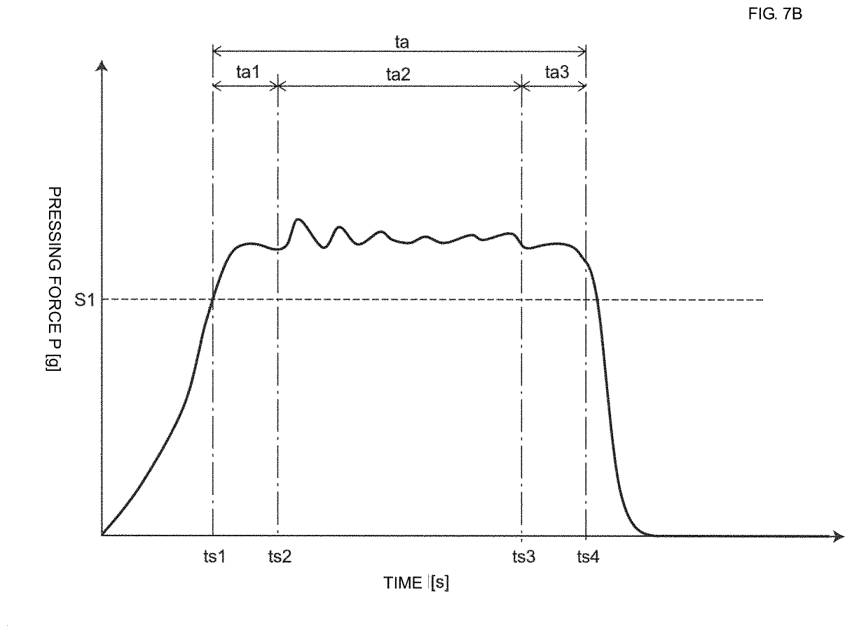

FIG. 7A is a diagram for illustrating an example of a method of calculating the average value Pz of the pressing forces P. As illustrated in FIG. 7A, the processor 21 starts the measurement process at time point ts1 when the pressing force P detected by the pressing force detection component 12 is equal to or greater than the first threshold value S1. The measurement process is performed during a predetermined time period ta. The predetermined time period ta is, for example, about 1.5 seconds.

The predetermined time period ta includes a first time period ta1 and a second time period ta2. The first time period ta1 ends at time point ts2 after a lapse of a predetermined time period from the time point ts1. The second time period ta2 ends at time point ts3 after a lapse of a predetermined time period from the time point ts2. The second time period ta2 is longer than the first time period ta1. For example, the first time period ta1 is about 0.5 seconds. The second time period ta2 is about 1.0 second.

The processor 21 calculates the average value Pz based on the values of the pressing forces P detected during the second time period ta2. This enables a more accurate calculation of the average value Pz of the pressing forces P. That is to say, the processor 21 does not use the values of the pressing forces P detected during the first time period ta1 immediately after the start of the measurement process for the calculation of the average value Pz. Compared with the first time period ta1, in the second time period ta2, the pressing forces P can be detected with stability. By calculating the average value Pz based on the values of the pressing forces P detected during the second time period ta2, a more accurate average value Pz of the pressing forces P can be calculated.

Note that the predetermined time period ta may further include a third time period ta3 after the second time period ta2.

FIG. 7B is a diagram for illustrating another example of the method of calculating the average value Pz of the pressing forces P. As illustrated in FIG. 7B, the predetermined time period ta includes a first time period ta1, a second time period ta2, and a third time period ta3. The first time period ta1 ends at time point ts2 after a lapse of a predetermined time period from the time point ts1. The second time period ta2 ends at time point ts3 after a lapse of a predetermined time period from the time point ts2. The third time period ta3 ends at time point ts4 after a lapse of a predetermined time period from the time point ts3. The second time period ta2 is longer than the first time period ta1 and the third time period ta3. The predetermined time period ta is, for example, about 2.0 seconds. For example, the first time period ta1 is about 0.5 seconds. The second time period ta2 is about 1.0 second. The third time period ta3 is about 0.5 seconds. The processor 21 calculates the average value Pz based on the values of the pressing forces P detected during the second time period ta2.

The processor 21 is arranged closer to the biosensor 11 than a center component C1 of the measurement device 1A in the lengthwise direction D1. Specifically, the processor 21 is arranged inside the probe 20. This suppresses the occurrence of noise.

The processor 21 can be realized using a semiconductor element and the like. The processor 21 can be formed using, for example, a microcomputer, a CPU, a MPU, a GPU, a DSP, a FPGA, an ASIC, a discrete semiconductor, a LSI, or the like. The functions of the processor 21 may be formed only using hardware or may be implemented by combining hardware and software. The processor 21 implements a predetermined function by reading out data or a program stored in a memory component of the processor 21, which is not illustrated in the drawing, and performing various arithmetic processes. The memory component can be realized, for example, by a hard disk (HDD), a SSD, a RAM, a DRAM, a ferroelectric memory, a flash memory, a magnetic disk, or a combination thereof.

### Operation Display Component

The operation display component 31 receives input from a user and displays information on the amount of the measuring target. For example, the operation display component 31 includes an operation component that receives input from a user and a display component that displays the information.

The operation includes one or a plurality of buttons that receive input from a user. The plurality of buttons includes, for example, a power button that switches between on and off of the power.

The display component displays information on the amount of the measuring target. The display component is, for example, a display. The information on the amount of the measuring target is transmitted, for example, from the calculation component included in the measurement device 1A to the display component. Alternatively, the information on the amount of the measuring target is transmitted from a calculation component included in another device different from the measurement device 1A to the display component via, for example, a network or the like.

The operation display component 31 is arranged on the top surface of the grip 30.

The measurement device 1A includes a control component that provides overall control of elements that make up the measurement device 1A. The control component includes, for example, a memory that stores a program therein and a processing circuit that corresponds to a processor such as a central processing unit (CPU) or the like. For example, in the control component, the processor executes the program stored in the memory. In embodiment 1, the control component controls the biosensor 11, the pressing force detection component 12, the processor 21, and the operation display component 31.

### Operation of Measurement Device

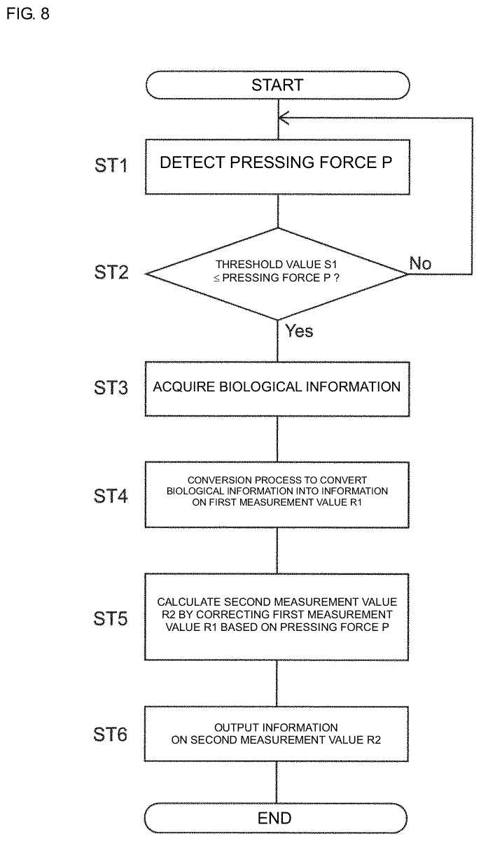

An example of operation of the measurement device 1A, that is, an example of a measurement method is described. FIG. 8 is a flowchart illustrating an example of operation of the measurement device 1A of embodiment 1 according to the present disclosure.

As illustrated in FIG. 8, in step ST1, the pressing force detection component 12 detects the pressing force P produced when the biosensor 11 comes into contact with a part of a living body to be measured. Specifically, a user brings the biosensor 11 arranged in the sensor 10 of the measurement device 1A into contact with a part to be measured in the oral cavity. In step ST1, the pressing force detection component 12 detects the pressing force P produced when the biosensor 11 is pressed against the part to be measured in the oral cavity. The information on the pressing force P detected by the pressing force detection component 12 is transmitted to the processor 21.

In step ST2, the processor 21 determines whether or not the pressing force P is equal to or greater than the first threshold value S1. In step ST2, the processor 21 receives the information on the pressing force P from the pressing force detection component 12. When the processor 21 determines that the pressing force P is equal to or greater than the first threshold value S1, the flow proceeds to step ST3. When the processor 21 determines that the pressing force P is less than the first threshold value S1, the flow returns to the step ST1.

In the step ST3, the biosensor 11 acquires biological information. The biological information acquired by the biosensor 11 is transmitted to the processor 21.

In embodiment 1, the biosensor 11 is an electrostatic capacitance sensor. The biosensor 11 acquires, as the biological information, information on the electrostatic capacitance. Furthermore, the biosensor 11 transmits the information on the electrostatic capacitance to the processor 21.

In step ST4, the processor 21 performs the conversion process that converts the biological information into information on the first measurement value R1. In embodiment 1, the processor 21 receives the information on the electrostatic capacitance from the biosensor 11 and converts the electrostatic capacitance into the frequency using the frequency conversion circuit.

In step ST5, the processor 21 calculates the second measurement value R2 by correcting the first measurement value R1 based on the pressing force P. The processor 21 determines the correction factor Q based on the pressing force P and calculates the second measurement value R2 by multiplying the first measurement value R1 by the correction factor Q. In embodiment 1, the processor 21 corrects the frequency by multiplying the frequency converted by the frequency conversion circuit by the correction factor Q. This enables the acquisition of the second measurement value R2.

In step ST6, the processor 21 outputs information on the second measurement value R2. For example, the processor 21 outputs the information on the second measurement value R2 to a calculation component included in the measurement device 1A. Alternatively, the processor 21 outputs the information on the second measurement value R2 to a calculation component included in another device different from the measurement device 1A.

The calculation component starts a calculation process to calculate the amount of the measuring target based on the information on the second measurement value R2. In embodiment 1, the amount of the measuring target is the amount of water content.

The information on the amount of the measuring target, which is calculated by the calculation component, is transmitted to the operation display component 31. The operation display component 31 displays the information on the amount of the measuring target.

As described above, by carrying out the steps ST1 to ST6, the measurement device 1A can output the information on the second measurement value R2 obtained by correcting the first measurement value R1 based on the pressing force P.

### Method of Using Measurement Device

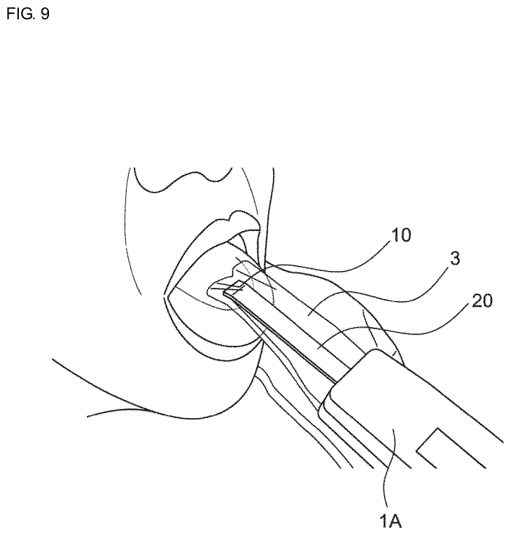

An example of a method of using the measurement device 1A is described using FIG. 9. FIG. 9 is a schematic view illustrating an example of a situation where the measurement device 1A of embodiment 1 according to the present disclosure is being used. Note that an exemplary method of using a device for measuring an inside of the oral cavity is described below as an example of the measurement device 1A.

As illustrated in FIG. 9, the sensor 10 and the probe 20 of the measurement device 1A are covered with a film 3. The power of the measurement device 1A is turned on by pressing a power button of the operation display component 31. This sets the measurement device 1A to the state where the measurement device 1A is ready for measurement.

During the measurement, the contact surface 10a of the measurement device 1A is brought into contact with a part to be measured in the oral cavity of a user. For example, the contact surface 10a is brought into contact with a tongue of a user.

In the measurement device 1A, the example of the operation illustrated in FIG. 8 is carried out.

The measurement device 1A detects the pressing force P using the pressing force detection component 12. The measurement device 1A starts the measurement process when the pressing force P detected by the pressing force detection component 12 is equal to or greater than the first threshold value. Whereas the measurement device 1A does not start the measurement process when the pressing force P detected by the pressing force detection component 12 is less than the first threshold value. In this case, the measurement device 1A may display an error indicating the inability to measure on the operation display component 31. Alternatively, the measurement device 1A may output sound information indicating the inability to measure. When the measurement device 1A does not starts a measurement, a user brings the contact surface 10a into contact with the tongue again.

After a lapse of a predetermined time period from the start of measurement, the measurement device 1A performs the conversion process that converts the biological information acquired by the biosensor 11 into the information on the first measurement value R1. The measurement device 1A calculates the second measurement value R2 by correcting the first measurement value R1 based on the pressing force P and transmits information on the second measurement value R2 to the calculation component. The calculation component calculates the amount of water content, as the amount of the measuring target, based on the information on the second measurement value R2.

When the measurement ends, the measurement device 1A displays the information on the amount of the measuring target as a measurement result on the operation display component 31. At this time, the measurement device 1A may notify a user of the end of measurement. For example, a message indicating the end of measurement may be displayed on the operation display component 31. Alternatively, the end of measurement may be notified to a user using sound information from a speaker.

### Advantageous Effects

The measurement device 1A according to embodiment 1 produces the following advantageous effects.

The measurement device 1A includes the biosensor 11, the pressing force detection component 12, and the processor 21. The biosensor 11 acquires the biological information. The pressing force detection component 12 detects the pressing force P produced when the biosensor 11 comes into contact with a part of a living body to be measured. The processor 21 calculates the second measurement value R2 by correcting, based on the pressing force P, the first measurement value R1 obtained based on the biological information and outputs the information on the second measurement value R2.

Such configuration improves measurement accuracy. According to the measurement device 1A, the measurement value can be corrected according to the size of the pressing force P produced when the biosensor 11 comes in contact with a part of a living body to be measured. This resolves the novel issue of variations in measurement value depending on the size of the pressing force P even when the biosensor 11 makes adequate contact with a part of a living body to be measured.

The pressing force P for pressing the biosensor 11 against the part to be measured varies depending on usage conditions, a user's skill level, and the like. The measurement device 1A facilitates an accurate measurement.

The processor 21 increases the correction amount of the first measurement value R1 as the pressing force P increases. Such configuration further improves the measurement accuracy.

The processor 21 starts the measurement process when the pressing force P is equal to or greater than the first threshold value S1. Such configuration starts the measurement when the biosensor 11 makes adequate contact with a part to be measured and further improves the measurement accuracy.

The processor 21 corrects the first measurement value R1 based on the average value Pz of the pressing forces P detected during a predetermined time period after the start of the measurement process. Such configuration further improves the measurement accuracy.

The processor 21 corrects the first measurement value R1 based on the average value Pz when the average value Pz of the pressing forces P is equal to or greater than the second threshold value S2 and equal to or less than the third threshold value S3. Such configuration corrects the first measurement value R1 based on the average value Pz of the pressing forces P when the biosensor 11 makes adequate contact with a part to be measured during the measurement. This further improves the measurement accuracy.

The measurement device 1A includes the housing 2 having the lengthwise direction D1 and storing therein the biosensor 11, the pressing force detection component 12, and the processor 21. The housing 2 has the sensor 10, the probe 20, and the grip 30. The sensor 10 is provided on the one end E1 side in the lengthwise direction D1. The grip 30 is provided on the other end E2 side in the lengthwise direction D1. The probe 20 is formed in a substantially rod-like shape and connects the sensor 10 and the grip 30. The biosensor 11 is arranged in the sensor 10. The pressing force detection component 12 is arranged in the sensor 10. The processor 21 is arranged in the probe 20. Such configuration facilitates the detection of the pressing force P produced when the biosensor 11 makes contact with a part to be measured. This further improves the measurement accuracy. Furthermore, by arranging the processor 21 in the probe 20, the occurrence of noise in the processor 21 can be suppressed.

The biosensor 11 has the detection surface 11 a that acquires biological information. The pressing force detection component 12 is arranged inside the sensor 10 and is arranged on the inner side of an outer perimeter of the detection surface 11a when viewed from the direction orthogonal to the detection surface 11a. Such configuration facilitates an accurate detection of the pressing force P produced when the biosensor 11 makes contact with a part to be measured. This further improves the measurement accuracy.

The biosensor 11 is an electrostatic capacitance sensor that detects electrostatic capacitance. The processor 21 performs a conversion process that converts the electrostatic capacitance detected by the electrostatic capacitance sensor into the frequency. Such configuration further improves the measurement accuracy.

The pressing force detection component 12 is a piezoelectric pressure sensor. Such configuration facilitates an accurate detection of the pressing force P produced when the biosensor 11 makes contact with a part to be measured. This further improves the measurement accuracy.

Note that in embodiment 1, the example is described in which the measurement device 1A includes the biosensor 11, the pressing force detection component 12, the processor 21, and the operation display component 31. However, the present embodiment is not limited this example. In the measurement device 1A, these elements may be realized using a single device or a plurality of devices. For example, the processor 21 and the operation display component 31 may be integrated with each other. The biosensor 11 and the processor 21 may be integrated with each other.

In embodiment 1, the example is described in which the operation display component 31 is provided in the measurement device 1A. However, the present embodiment is not limited to this example. The operation display component 31 may not be provided in the measurement device 1A. For example, the operation display component 31 may be provided in another device different from the measurement device 1A.

In embodiment 1, the example is described in which the measurement device 1A is the device for measuring an inside of the oral cavity and the amount of water content is measured as the amount of the measuring target. However, the present embodiment is not limited to this example. For example, the measurement device 1A may measure a saliva secretion volume, a bite force, a tongue pressure force, a tongue color tone, and/or amounts of various substances contained in saliva. Specifically, the measurement device 1A may measure, as the measuring target, the amount of secreted electrolyte, various enzymes, protein, ammonia, or the like.

Alternatively, the measurement device 1A may be a pulse meter, a pulse oximeter, or the like.

In embodiment 1, the example is described in which the housing 2 includes the sensor 10, the probe 20, and the grip 30. However, the present embodiment is not limited to this example.

In embodiment 1, the example is described in which the biosensor 11 is an electrostatic capacitance sensor. However, the present embodiment is not limited to this example. The biosensor 11 may be any sensor that can acquire biological information. For example, the biosensor 11 may be at least one of an impedance measurement sensor, a load sensor, and a moisture sensor.

In embodiment 1, the example is described in which the detection surface 11a of the biosensor 11 is formed in a substantially rectangular shape when viewed from the height direction (Z direction) of the measurement device 1A. However, the present embodiment is not limited to this example. For example, the detection surface 11a of the biosensor may have a substantially polygonal shape, a substantially circular shape, or a substantially elliptic shape when viewed from the height direction (Z direction) of the measurement device 1A.

In embodiment 1, the example is described in which the pressing force detection component 12 is arranged in the sensor 10. However, the present embodiment is not limited to this example. The pressing force detection component 12 may be arranged at any location, provided that the pressing force detection component 12 can detect the pressing force P produced when the biosensor 11 comes into contact with a part to be measured.

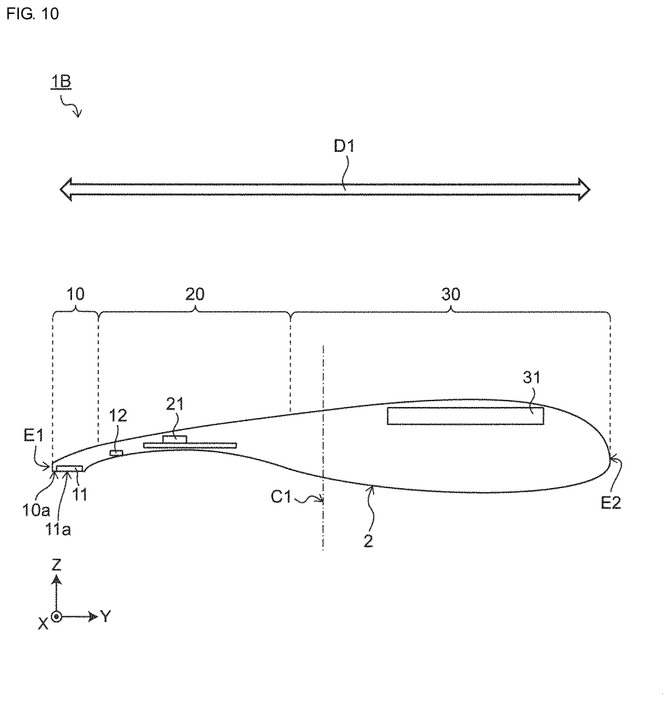

FIG. 10 is a view illustrating an internal configuration of a measurement device 1B of a modified example of embodiment 1 according to the present disclosure. As illustrated in FIG. 10, in the measurement device 1B, the pressing force detection component 12 may be arranged in the probe 20. Such configuration also enables the pressing force detection component 12 to facilitate the detection of the pressing force P.

In embodiment 1, the example is described in which the measurement device 1A includes a single pressing force detection component 12. However, the present embodiment is not limited to this example. The measurement device 1A may include one or a plurality of the pressing force detection components 12.

In embodiment 1, the example is described in which the processor 21 corrects the first measurement value R1 based on the average value Pz of the pressing forces P detected during a predetermined time period. However, the present embodiment is not limited to this example. For example, the processor 21 may correct the first measurement value R1 based on a median value of the pressing forces P detected during a predetermined time period.

In embodiment 1, the example is described in which the processor 21 includes the conversion circuit that performs the conversion process that converts the electrostatic capacitance into the frequency. However, the present embodiment is not limited to this example. The processor 21 may include a circuit that converts the biological information acquired by the biosensor 11 into information other than the frequency. Alternatively, the processor 21 may not need to include the conversion circuit. In this case, the processor 21 may directly use the biological information as the first measurement value R1.

In embodiment 1, the example is described in which the operation display component 31 includes the operation component and the display component. However, the present embodiment is not limited to this example. The operation display component 31 may only be necessary to include at least one of the operation component and the display component.

In embodiment 1, an example of the operation of the measurement device 1A is described using the steps ST1 to ST6 illustrated in FIG. 8. However, the present embodiment is not limited to this example. For example, the steps ST1 to ST6 illustrated in FIG. 8 may be integrated or divided. Alternatively, the flowchart illustrated in FIG. 8 may include an additional step. For example, a step for displaying a measurement result on the operation display component 31 may be added. The order of carrying out the steps ST1 to ST6 is also not limited to the one illustrated in FIG. 8.

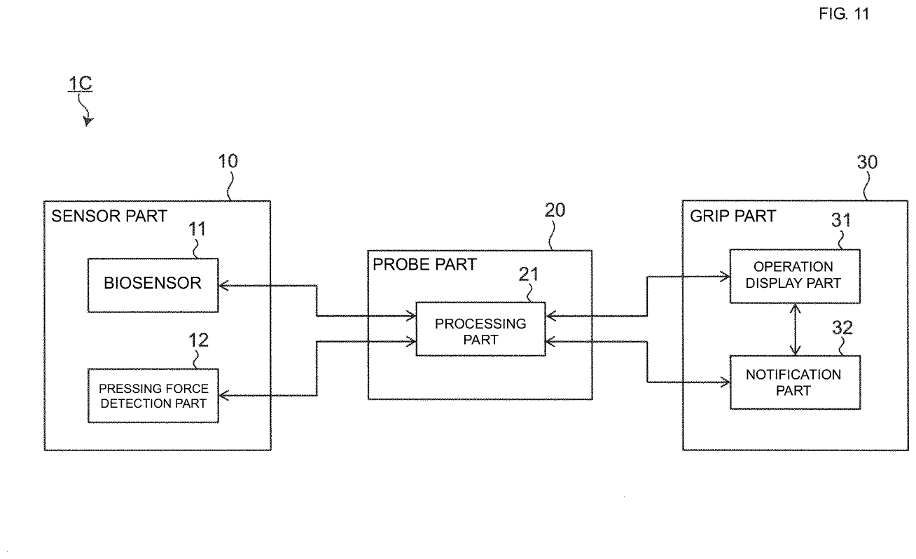

FIG. 11 is a block diagram illustrating a schematic configuration of a measurement device 1C of a modified example of embodiment 1 according to the present disclosure. As illustrated in FIG. 11, the measurement device 1C includes a notification component 32 that gives notice of information. For example, the notification component 32 is a device that outputs sound information and/or optical information. For example, the notification component 32 may be a speaker, a LED, a display, or the like. The notification component 32 may output information that give notice of the end of measurement and information that gives notice of a measurement error. The notification component is controlled by the control component.

For example, the processor 21 determines whether or not the pressing force P is in the range between predetermined threshold values and transmits information on the determination result to the notification component 32. The notification component 32 outputs information based on the information on the determination result. For example, when the pressing force P is in the range between predetermined threshold values, the notification component 32 outputs information that gives notice of the end of measurement. Alternatively, when the pressing force P is out of the range between predetermined threshold values, the notification component 32 outputs information that gives notice of a measurement error. Such configuration improves usability of the measurement device 1C.

### Embodiment 2

A measurement device according to embodiment 2 of the present disclosure is described. Note that in embodiment 2, features different from those of embodiment 1 are mainly described. In embodiment 2, elements identical or corresponding to those elements of embodiment 1 are described using the same reference codes. Furthermore, in embodiment 2, the descriptions overlapping with embodiment 1 are omitted.

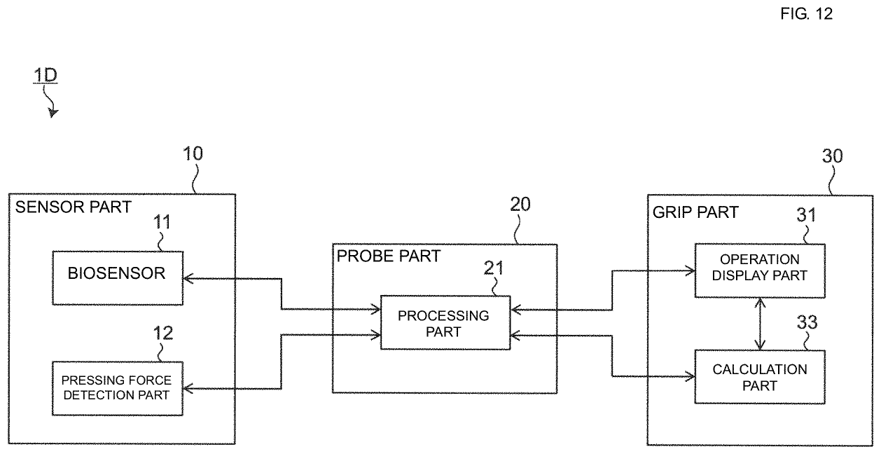

An example of a measurement device of embodiment 2 is described using FIG. 12. FIG. 12 is a block diagram illustrating a schematic configuration of an example of a measurement device 1D of embodiment 2 according to the present disclosure.

Embodiment 2 is different from embodiment 1 in including a calculation component 33.

As illustrated in FIG. 12, the measurement device 1D includes the calculation component 33. The calculation component 33 calculates the amount of a measuring target based on the second measurement value R2 calculated in the processor 21.

The calculation component 33 is stored in the grip 30 of the housing 2. The calculation component 33 receives information on the second measurement value R2 from the processor 21. The calculation component 33 calculates the amount of the measuring target based on the received information on the second measurement value R2. In embodiment 2, the information on the second measurement value R2 is frequency information. The calculation component 33 calculates the amount of water content based on the frequency information. The calculation component 33 is controlled by the control component.

The calculation component 33 can be realized using a semiconductor element and the like. The functions of the calculation component 33 may be formed only using hardware or may be implemented by combining hardware and software. The calculation component 33 includes, for example, a water content amount calculation circuit that calculates the amount of water content based on the amount of change in frequency. Note that the amount of change in frequency is a difference between a reference frequency and a frequency converted by the processor 21 based on information on the electrostatic capacitance. The reference frequency means a frequency in a standard air atmosphere.

The calculation component 33 includes a memory component. The memory component can be realized, for example, by a hard disk (HDD), a SSD, a RAM, a DRAM, a ferroelectric memory, a flash memory, a magnetic disk, or a combination thereof. For example, when carrying out the calculation of the amount of the measuring target, the calculation component 33 stores, in the memory component, the information on the second measurement value R2 transmitted from the processor 21.

The information on the amount of water content calculated by the calculation component 33 is transmitted to the operation display component 31.

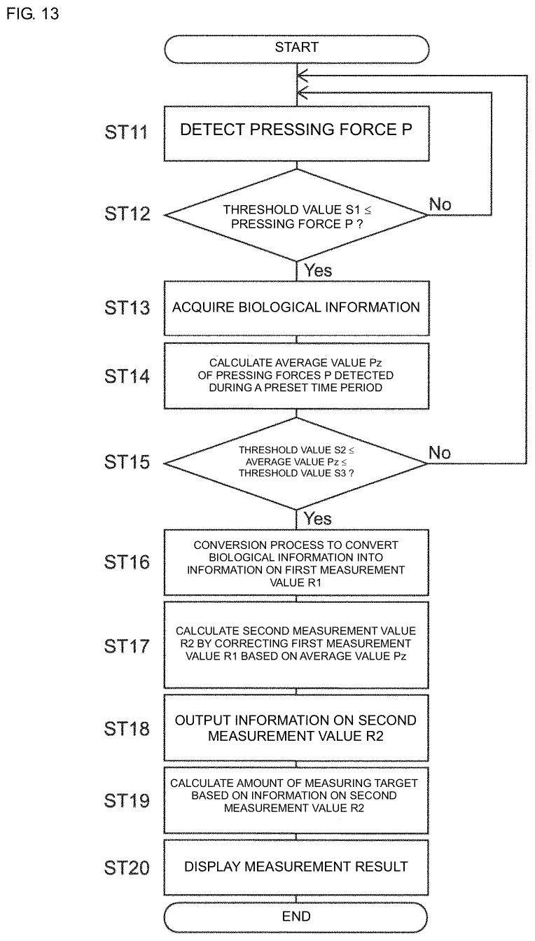

FIG. 13 is a flowchart illustrating an example of operation of the measurement device 1D of embodiment 2 according to the present disclosure. Steps ST11 to ST13 and ST16 to ST18 illustrated in FIG. 13 are substantially the same as the steps ST1 to ST6 illustrated in FIG. 8 of embodiment 1, and thus detailed descriptions thereof are omitted.

As illustrated in FIG. 13, in step ST11, the pressing force detection component 12 detects the pressing force P.

In step ST12, the processor 21 determines whether or not the pressing force P is equal to or greater than the first threshold value S1. When the processor 21 determines that the pressing force P is equal to or greater than the first threshold value S1, the flow proceeds to step ST13. When the processor 21 determines that the pressing force P is less than the first threshold value S1, the flow returns to the step ST11.

In the step ST13, the biosensor 11 acquires biological information.

In step ST14, the processor 21 calculates the average value Pz of the pressing forces P detected during the predetermined time period. Note that the method of calculating the average value Pz of the pressing forces P is substantially the same as that of embodiment 1, and thus the description thereof is omitted.

In step ST15, the processor 21 determines whether or not the average value Pz of the pressing forces P is equal to or greater than the second threshold value S2 and equal to or less than the third threshold value S3. When the processor 21 determines that the average value Pz is equal to or greater than the second threshold value S2 and equal to or less than the third threshold value S3, the flow proceeds to step ST16. When the processor 21 determines that the average value Pz is equal to or less than the second threshold value S2 or equal to or greater than the third threshold value S3, the flow returns to the step ST11.

In the step ST16, the processor 21 performs the conversion process that converts the biological information into information on the first measurement value R1.

In step ST17, the processor 21 calculates the second measurement value R2 by correcting the first measurement value R1 based on the average value Pz of the pressing forces P.

In step ST18, the processor 21 outputs information on the second measurement value R2. The processor 21 outputs the information on the second measurement value R2 to the calculation component 33.

In step ST19, the calculation component 33 calculates the amount of the measuring target based on the information on the second measurement value R2. The calculation component 33 receives the information on the second measurement value R2 from the processor 21 and calculates the amount of the measuring target based on the second measurement value R2. Information on the calculated amount of the measuring target is transmitted to the operation display component 31.

In step ST20, the operation display component 31 displays a measurement result. The operation display component 31 receives the information on the amount of the measuring target from the calculation component 33 and display the information on the amount of the measuring target.

As described above, by carrying out the steps ST11 to ST20, the measurement device 1D can calculate the amount of the measuring target.

### Advantageous Effects

The measurement device 1D according to embodiment 2 produces the following advantageous effects.

The measurement device 1D includes the calculation component 33 that calculates the amount of the measuring target based on the second measurement value R2. Such configuration enables the calculation of the amount of the measuring target.

Note that in embodiment 2, the example is described in which the calculation component 33 is arranged inside the grip 30. However, the present embodiment is not limited to this example. For example, the calculation component 33 may be arranged inside the probe 20. In this case, the calculation component 33 and the processor 21 may be integrated with each other.

In embodiment 2, the example is described in which the calculation component 33 calculates the amount of water content as the amount of the measuring target. However, the present embodiment is not limited to this example. Furthermore, the example is described in which the calculation component 33 includes the water content amount calculation circuit that calculates the amount of water content based on the amount of change in frequency. However, the present embodiment is not limited to this example. For example, the calculation component 33 may only be necessary to include the calculation circuit that calculates the amount of the measuring target.

### Embodiment 3

A measurement system according to embodiment 3 of the present disclosure is described. Note that in embodiment 3, features different from those of embodiment 1 are mainly described. In embodiment 3, elements identical or corresponding to those of embodiment 1 are described using the same reference codes. Furthermore, in embodiment 3, the descriptions overlapping with embodiment 1 are omitted.

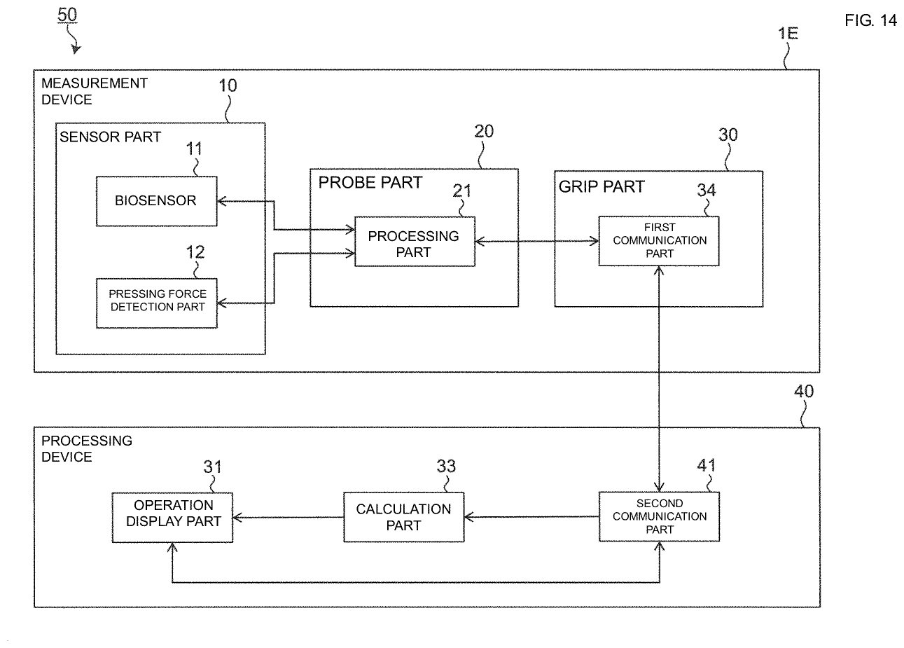

An example of a measurement system of embodiment 3 is described using FIG. 14. FIG. 14 is a block diagram illustrating a schematic configuration of an example of a measurement system 50 of embodiment 3 according to the present disclosure.

Embodiment 3 is different from embodiment 1 in that information acquired by a measurement device 1E is transmitted to a processing device 40 and the processing device 40 calculates the amount of a measuring target.

As illustrated in FIG. 14, the measurement system 50 includes the measurement device 1E that makes contact with a part of a living body to be measured and the processing device 40 that communicates with the measurement device 1E.

### Measurement Device

The measurement device 1E includes the biosensor 11, the pressing force detection component 12, the processor 21, and a first communication component 34. In embodiment 3, the biosensor 11, the pressing force detection component 12, and the processor 21 are substantially the same as those of embodiment 1, and thus the descriptions thereof are omitted.

The first communication component 34 communicates with the processing device 40. Specifically, the first communication component 34 transmits the information on the second measurement value R2 output from the processor 21 to the processing device 40.

The first communication component 34 includes a circuit that conforms to predetermined communication standards and communicates with the processing device 40. The predetermined communication standards include, for example, LAN, Wi-Fi (Registered Trademark), Bluetooth (Registered Trademark), USB, HDMI (Registered Trademark), controller area network (CAN), serial peripheral interface (SPI), universal asynchronous receiver/transmitter (UART), and inter-integrated circuit (I2C).

The measurement device 1E includes a first control component that provides overall control for elements that make up the measurement device 1E. The first control component includes, for example, a memory that stores a program therein and a processing circuit that corresponds to a processor such as a central processing unit (CPU) or the like. For example, in the first control component, the processor executes the program stored in the memory. In embodiment 3, the first control component controls the biosensor 11, the pressing force detection component 12, the processor 21, and the first communication component 34.

### Processing Device

The processing device 40 receives information from the measurement device 1E and calculates the amount of the measuring target based on the received information. Specifically, the processing device 40 receives the information on the second measurement value R2 from the measurement device 1E and calculates the amount of the measuring target based on the second measurement value R2.

The processing device 40 is a computer. For example, the processing device 40 may be a mobile terminal such as a smartphone, a tablet terminal, or the like. Alternatively, the processing device 40 may be a server connected to a network.

The processing device 40 includes a second communication component 41, the operation display component 31, and the calculation component 33. In embodiment 3, the operation display component 31 and the calculation component 33 are substantially the same as those of embodiment 1 and embodiment 2, and thus the descriptions thereof are omitted.

The second communication component 41 communicates with the measurement device 1E. Specifically, the second communication component 41 receives information on the second measurement value R2 from the first communication component 34 of the measurement device 1E.

The second communication component 41 includes a circuit that conforms to predetermined communication standards and communicates with the measurement device 1E. The predetermined communication standards include, for example, LAN, Wi-Fi (Registered Trademark), Bluetooth (Registered Trademark), USB, HDMI (Registered Trademark), controller area network (CAN), serial peripheral interface (SPI), universal asynchronous receiver/transmitter (UART), and inter-integrated circuit (I2C).

The processing device 40 receives the information on the second measurement value R2 from the measurement device 1E via the second communication component 41.

In the processing device 40, the calculation component 33 calculates the amount of the measuring target based on the information on the second measurement value R2 received from the measurement device 1D. In embodiment 3, the calculation component 33 calculates the amount of water content based on the information on the second measurement value R2. The information on the calculated amount of water content is transmitted to the operation display component 31. The operation display component 31 displays the information on the calculated amount of water content.

The processing device 40 includes a second control component that provides overall control for elements that make up the processing device 40. The second control component includes, for example, a memory that stores a program therein and a processing circuit that corresponds to a processor such as a central processing unit (CPU) or the like. For example, in the second control component, the processor executes the program stored in the memory. In embodiment 3, the second control component controls the second communication component 41, the operation display component 31, and the calculation component 33.

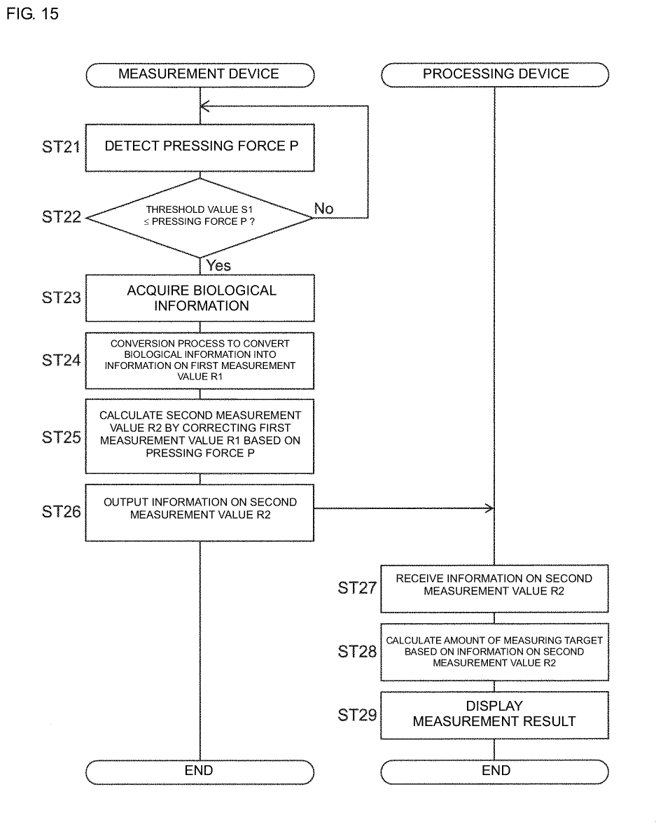

FIG. 15 is a flowchart illustrating an example of operation of the measurement system 50 of embodiment 3 according to the present disclosure. Steps ST21 to ST26 illustrated in FIG. 15 are substantially the same as the steps ST1 to ST6 illustrated in FIG. 8 of embodiment 1, and thus detailed descriptions thereof are omitted.

As illustrated in FIG. 15, in step ST21, the pressing force detection component 12 detects the pressing force P.

In step ST22, the processor 21 determines whether or not the pressing force P is equal to or greater than the first threshold value S1. When the processor 21 determines that the pressing force P is equal to or greater than the first threshold value S1, the flow proceeds to step ST23. When the processor 21 determines that the pressing force P is less than the first threshold value S1, the flow returns to the step ST21.

In the step ST23, the biosensor 11 acquires biological information. The biological information acquired by the biosensor 11 is transmitted to the processor 21.

In step ST24, the processor 21 performs the conversion process that converts the biological information into information on the first measurement value R1.

In step ST25, the processor 21 calculates the second measurement value R2 by correcting the first measurement value R1 based on the pressing force P.

In step ST26, the processor 21 outputs information on the second measurement value R2. The processor 21 transmits the information on the second measurement value R2 to the processing device 40 using the first communication component 34.

In step ST27, the second communication component 41 receives the information on the second measurement value R2. The information on the second measurement value R2 received by the second communication component 41 is transmitted to the calculation component 33.

In step ST28, the calculation component 33 calculates the amount of the measuring target based on the information on the second measurement value R2. In embodiment 3, the calculation component 33 calculates the amount of water content as the amount of the measuring target. The calculation component 33 transmits information on the calculated amount of the measuring target to the operation display component 31.

In step ST29, the operation display component 31 displays a measurement result.

As described above, by carrying out the steps ST21 to ST29, the measurement system 50 can calculate the amount of the measuring target.

### Advantageous Effects

The measurement system 50 according to embodiment 3 produces the following advantageous effects.

The measurement system 50 includes the measurement device 1E and the processing device 40 that communicates with the measurement device 1E. The measurement device 1E includes the biosensor 11, the pressing force detection component 12, the processor 21, and the first communication component 34. The biosensor 11 acquires biological information. The pressing force detection component 12 detects the pressing force P produced when the biosensor 11 comes into contact with a part of a living body to be measured. The processor 21 calculates the second measurement value R2 by correcting the first measurement value R1, which is obtained based on the biological information, based on the pressing force P and outputs information on the second measurement value R2. The first communication component 34 transmits the information on the second measurement value R2 to the processing device 40. The processing device 40 includes the second communication component 41 and the calculation component 33. The second communication component 41 receives the information on the second measurement value R2 from the first communication component 34 of the measurement device 1E. The calculation component 33 calculates the amount of the measuring target based on the information on the second measurement value R2.

Such configuration improves measurement accuracy. According to the measurement system 50, the measurement value can be corrected according to the size of the pressing force P produced when the biosensor 11 comes in contact with a part of a living body to be measured. This resolves the novel issue of variations in measurement value depending on the size of the pressing force P even when the biosensor 11 makes adequate contact with a part of a living body to be measured.

The pressing force P for pressing the biosensor 11 against the part to be measured varies depending on usage conditions, a user's skill level, and the like. The measurement system 50 facilitates an accurate measurement.

Note that in embodiment 3, the example is described in which the processing device 40 includes the operation display component 31. However, the present embodiment is not limited to this example. In the processing device 40, the operation display component 31 is not an essential element. For example, the operation display component 31 may be provided in the measurement device 1E. Alternatively, the operation display component 31 may be provided in another external device.

In embodiment 3, the example is described in which the measuring target of the measurement system 50 is water content. However, the present embodiment is not limited to this example. The measurement system 50 may only be necessary to measure the amount of a measuring target.

In embodiment 3, the example is described in which the measurement system 50 includes the measurement device 1E. However, the present embodiment is not limited to this example.

With regard to preferred embodiments, the present disclosure is sufficiently described with reference to the accompanying drawings. However, various variations and modifications are apparent to those skilled in the art. It is to be understood that such variations and modifications are included within the scope of the present disclosure, provided that such variations and modifications do not deviate from the scope of the present disclosure described by the attached claims.

The measurement devices and the measurement system of the present disclosure are applicable to, for example, a water content amount measurement device that measures the amount of water content in the oral cavity and other similar devices.

While preferred embodiments of the invention have been described above, it is to be understood that variations and modifications will be apparent to those skilled in the art without departing from the scope and spirit of the invention. The scope of the invention, therefore, is to be determined solely by the following claims.

1. A measurement device comprising:
a biosensor that acquires biological information;
a pressing force detection component that detects a pressing force produced when the biosensor makes contact with a part of a living body to be measured; and
a processor that:
converts the biological information to a first measurement value;
calculates a second measurement value by correcting the first measurement value based on the pressing force; and
outputs the second measurement value.

2. The measurement device according to claim 1, wherein the processor calculates the second measurement value by increasing the first measurement value by a correction amount as the pressing force increases.

3. The measurement device according to claim 1, wherein the processor starts a measurement process when the pressing force is equal to or greater than a first threshold value.

4. The measurement device according to claim 1, wherein the processor corrects the first measurement value based on an average value of pressing forces detected during a predetermined time period after the start of a measurement process.

5. The measurement device according to claim 4, wherein the processor corrects the first measurement value based on the average value when the average value is equal to or greater than a second threshold value and equal to or less than a third threshold value.

6. The measurement device according to claim 1, further comprising:
a housing storing therein the biosensor, the pressing force detection component, and the processor, wherein the housing includes:
a sensor disposed on a first one end along a lengthwise direction of the housing,
a grip provided on a second end along the lengthwise direction that is opposite the first end, and
a probe having a rod-like shape, the probe connecting the sensor and the grip;
wherein,
the biosensor is disposed in the sensor,
the pressing force detection component is disposed in the sensor or the probe, and
the processor is disposed in the probe.

7. The measurement device according to claim 6, wherein
the biosensor has a detection surface that acquires the biological information, and
the pressing force detection component is disposed inside the sensor and on an inner side of an outer perimeter of the detection surface when viewed from a direction orthogonal to the detection surface.

8. The measurement device according to claim 1, wherein
the biosensor is an electrostatic capacitance sensor that detects electrostatic capacitance, wherein the electrostatic capacitance is the biological information; and
the processor is configured to convert the electrostatic capacitance detected by the electrostatic capacitance sensor into a frequency, wherein the frequency is the first measurement value.

9. The measurement device according to claim 1, further comprising:
a calculation component that calculates an amount of a measuring target based on the second measurement value.

10. The measurement device according to claim 9, wherein the amount of the measuring target is an amount of water content.

11. The measurement device according to claim 1, wherein the pressing force detection component is a piezoelectric pressure sensor.

12. The measurement device according to claim 1, further comprising:
a notification component that gives notice of information, wherein the processor determines whether or not the pressing force is in a predetermined range and outputs information on a determination result to the notification component.

13. The measurement device according to claim 1, wherein the part of the living body to be measured is a part in an oral cavity.

14. A measurement system comprising:
a measurement device; and
a processing device that communicates with the measurement device;
wherein the measurement device includes:
a biosensor that acquires biological information,
a pressing force detection component that detects a pressing force produced when the biosensor contacts a part of a living body to be measured,
a processor that:
converts the biological information to a first measurement value;
calculates a second measurement value by correcting the first measurement value based on the pressing force; and
a first communication component that transmits the second measurement value to the processing device, and
wherein the processing device includes:
a second communication component that receives the second measurement value from the first communication component of the measurement device, and
a calculation component that calculates an amount of a measuring target based on the second measurement value.

15. A measurement device comprising:
a housing storing therein a biosensor, a pressing force detection component, and a processor, wherein the housing includes:
a sensor disposed on a first end along a lengthwise direction of the housing,
a grip disposed on a second end along the lengthwise direction that opposes the first end, and
a probe having a rod-like shape, the probe connecting the sensor and the grip;
wherein the biosensor is disposed in the sensor, the pressing force detection component is disposed in the sensor or the probe, and the processor is disposed in the probe.

16. The measurement device of claim 15, wherein:
the biosensor is configured to acquire biological information;
the pressing force detection component is configured to detect a pressing force produced when the biosensor makes contact with a part of a living body to be measured; and
the processor is configured to:
convert the biological information to a first measurement value;
calculate a second measurement value by correcting the first measurement value based on the pressing force; and
output the second measurement value.

17. The measurement device according to claim 16, wherein:
the biosensor is an electrostatic capacitance sensor that detects electrostatic capacitance, wherein the electrostatic capacitance is the biological information; and
the processor is configured to convert the electrostatic capacitance detected by the electrostatic capacitance sensor into a frequency, wherein the frequency is the first measurement value.

18. The measurement device according to claim 16, further comprising a calculation component that calculates an amount of a measuring target based on the second measurement value.

19. The measurement device according to claim 18, wherein the amount of the measuring target is an amount of water content.

20. The measurement device according to claim 15, wherein:
the biosensor has a detection surface that acquires biological information, and
the pressing force detection component is disposed inside the sensor and is disposed on an inner side of an outer perimeter of the detection surface when viewed from a direction orthogonal to the detection surface.
